(19) United States
(12) Patent Application Publication
(10) Pub. No.: US 2010/0034878 A1
(43) Pub. Date: Feb. 11, 2010

(54) COMBINATION OF VITAMIN K AND VITAMIN K ANTAGONIST SUCH AS R-ISOMER OF WARFARIN, PHENPROCOUMON OR R-ISOMER OF PHENPROCOUMON AS ANTICOAGULANT THERAPY

(76) Inventors: Henry I. Bussey, Lakehills, TX (US); Marie B. Walker, Lakehills, TX (US)

Correspondence Address: JACKSON WALKER LLP, 901 MAIN STREET, SUITE 6000, DALLAS, TX, 75202-3797, US

(21) Appl. No.: 12/519,505

(22) PCT Filed: Dec. 18, 2007
(86) PCT No.: PCT/US2007/025889

§ 371 Date: Sep. 8, 2009

Related U.S. Application Data
(60) Provisional application No. 60/875,715, filed on Dec. 19, 2006.

(51) Int. Cl. A61K 9/28; A61K 31/122; A61K 31/35

(52) U.S. Cl. 424/474; 514/682; 514/457

(57) ABSTRACT

Medications which are derived by combining one or more doses of the agonist, vitamin K, with doses of one or more selected vitamin K antagonists such as the R-isomer of warfarin, the R-isomer of phenprocoumon, or the racemic phenprocoumon are useful in anticoagulant therapy. These products are designed to minimize or circumvent the detrimental impact of newly identified genetic-based variables that currently significantly complicate the therapeutic response of individual patients to anticoagulation therapy.

### BACKGROUND

This application claims priority to U.S. Provisional Patent Application Ser. No. 60/875,715, entitled “New Oral Anticoagulant Formulations,” filed on Dec. 19, 2006, the entire content of which is hereby incorporated by reference.

This invention pertains to compositions for use in oral anticoagulant therapy that comprise low doses of vitamin K with one or more vitamin K antagonists.

Various vitamin K antagonists (“VKAs”) are used worldwide by millions of people to prevent various blood clotting problems such as heart attack, stroke, pulmonary embolism, prosthetic heart valve thrombosis and others. VKAs exert their effect by interfering with the ability of the liver to use vitamin K in the synthesis of various functional blood proteins that are required for the body to form a blood clot. These functional blood clotting proteins are referred to as factor II, factor VII, factor IX, and factor X (where II, VII, IX, and X are the Roman numerals for 2, 7, 9 and 10). Both the efficacy (prevention of blood clots) and safety (bleeding risks) of VKAs are strongly correlated with the extent to which such therapy achieves the desired INR value (INR=International Normalized Ration, which is the correct method to report the blood clotting test known as the prothrombin time). If the INR increases above the desired range, catastrophic and even fatal bleeding may occur. If the INR falls below the desired range, the beneficial effects of this therapy rapidly decline and he patient is placed at an increased risk of an unwanted blood clot. For example, in the recent analysis of over 3,000 patients taking warfarin, the rates of stroke, heart attack, death, and major hemorrhage appeared to be reduced by at least 50% in those patients whose INRs were in the desired range 83% of the time, compared to those whose INRs were in range approximately 50% of the time (White, et al. 2007). Therefore, it is highly desirable and beneficial to find a way to safely and effectively improve the stability of INR control in such patients.

A variety of factors such as drug interactions, changes in diet, alcohol consumption, concomitant diseases, changes in physical activity, and other factors can significantly alter the response to available VKAs and thereby induce dangerous changes in the INR. The impact of these various interacting factors has been most studied with warfarin, which is the only VKA available in North America and is the most commonly used VKA world wide. U.S. Pat. No. 2,427,578 describes the chemical composition for warfarin. Only recently has data become available to explain why some individuals are unusually sensitive to this VKA and why others tend to metabolize the drug more slowly and, therefore, accumulate excessive amounts of the medication. The successful identification of genetic polymorphisms that influence both sensitivity to warfarin and its rate of metabolism has provided avenues for the development of new medications that are not subject to these confounding influences.

The genetic polymorphisms that influence the rate at which warfarin is metabolized occur in the 2C9 enzyme of the hepatic cytochrome P-450 enzyme system (“CYP 2C9”). Five different polymorphisms have been identified in addition to the “wild type” (i.e., the usual or typical form of CYP 2C9). The 5 different aberrant CYP 2C9 polymorphisms each induce different degrees of reduction in the metabolism of the warfarin. The reduced rates of metabolism result in a longer half-life of the drug which, in turn, means that at any given dose, patients with aberrant CYP 2C9 polymorphisms will have drug half-lives that are variably longer than usual. The longer the half-life, the longer the patient will accumulate drug in the plasma until a steady state is finally reached. Consequently, patients with these aberrant CYP 2C9 polymorphisms are at greater risk of accumulating excessive amounts of the VKA and, therefore, are at greater risk of bleeding. In addition to the fact that CYP 2C9 genetic polymorphisms help explain variations in the metabolic clearance of warfarin, these variations also help explain why the manifestations of various drug interactions can be so variable and unpredictable; or why some individuals appear to be more susceptible to drug interactions than others. Many, and probably most, of the most significant drug interactions are mediated through either stimulation or inhibition of the metabolic activity of the CYP 2C9 enzyme system. Further, alterations in the INR due to alcohol, smoking, and other factors also are likely mediated by altering the activity of the CYP 2C9.

The genetic polymorphisms that influence the individual patient's sensitivity to the VKAs occur in the gene that codes for the enzyme vitamin K epoxide reductase (“VKORC1”). This VKORC1 gene was identified in 2003 and, subsequently, it was realized that there are 3 clinically important polymorphisms that determine whether a patient is resistant to the effects of VKAs, has a “typical” sensitivity to VKAs, or is unusually sensitive to VKAs. Those who are resistant require larger doses of the VKA, and high blood concentrations of the VKA, in order to achieve a therapeutic INR. By contrast, those with the “sensitive” genotype will demonstrate an early and abrupt increase in the INR with only a modest dose of a VKA.

The clinical importance of this information regarding the CYP 2C9 and VKORC1 genetic polymorphism led the U.S. Food and Drug Administration (FDA) to modify the package insert for warfarin this year. Specifically, the change in the package insert is designed to make clinicians aware of the potential value of genetic testing in patients who are about to start warfarin therapy. Further, with input from the FDA, the American Enterprise Institute—Brookings Joint Center recently reported their estimate that genetic testing for such patients in the U.S. would avoid 85,000 serious bleeding events and 17,000 strokes annually. They further estimated that such genetic testing would reduce health care costs by $1.1 billion dollars annually. However, these projections have been challenged by some as unfounded and one should realize that each test (CYP 2C9 and VKORC1) is estimated to cost about $250 each, or $500 for both tests. Considering that between 2 million and 3 million US citizens take warfarin, the cost of genetic testing for these patients would be approximately $1 to $1.5 billion.

U.S. Pat. No. 5,041,430 pertains to the combination of low does of an antiocoagulant, such as warfarin, and an antiplatelet agent, such as aspirin. The disclosures of this patent do not take into account any genetic sensitivities to VKAs and do not describe any suitable ways to avoid adverse effects.

Canadian Patent No. 2388103 discusses the concept of combing vitamin K with various racemic VKAs, such as dicoumarol, acenocoumarol, phenprocoumon, and warfarin, to counteract dietary fluctuations, but it does not take into account any genetic sensitivities to VKAs (such as the VKORC1 genetic polymorphisms) and it does not discuss the feasibility or advantages of using individual isomers of the VKAs.

### SUMMARY

The present invention pertains to compositions for use in oral anticoagulant therapy that comprise both vitamin K in low dosage and one or more vitamin K antagonists, such as the R-isomer of warfarin, the R-isomer of phenprocoumon, or racemic phenprocoumon. The composition is administered in a single dosage.

The current compositions can be used as an alternative, or in addition, to genetic testing and will allow one to circumvent or greatly reduce the role of the six CYP 2C9 polymorphisms in altering the response to VKAs. These compositions will minimize the effect of the three different VKORC1 polymorphisms on the response to VKAs. The end result will be combination product anticoagulant medications with more predictable dose response relationships because these products will avoid or minimize the effect of CYP 2C9 genetic polymorphisms, reduce the effect of VKORC1 genetic polymorphisms, avoid or minimize the impact many of the more substantial drug interactions, and reduce the effect of transient dietary changes. The new medications will allow for more effective and safer therapy that can be managed more easily than the currently available agents. These new medications will be achieved by combining the agonist, vitamin K, in low doses with selected VKAs. It is necessary to combine the two products (the low dose vitamin K and the selected VKAs) in the same dosage formulation because discontinuation of either agent without discontinuation of the other agent would increase the risk of thrombosis or bleeding for the patient.

The present invention pertains to compositions for use in oral anticoagulant therapy comprising low doses of vitamin K in combination with a vitamin K antagonist. The vitamin K antagonist may be the R isomer of warfarin, the R isomer of phenprocoumon, or a racemic phenprocoumon mixture.

The R-isomer of warfarin is selected because it is not metabolized by the 2C9 enzyme. Instead, the R-isomer is metabolized by several other hepatic enzymes such as 3A4, 1A1, and 1A2. By contrast, the S-isomer is almost totally metabolized by the 2C9 enzyme. Use of the R-isomer of warfarin, therefore, avoids the variable response induced by the CYP 2C9 polymorphisms and eliminates the mechanism of many of the most severe warfarin drug interactions.

Racemic phenprocoumon and the R-isomer of phenprocoumon are selected because although both isomers are metabolized by 2C9, the role of 2C9 in the metabolism of phenprocoumon is quite limited.

The present invention also utilizes a low dose of vitamin K in order to (1) increase the overall level of vitamin K in the body, such that changes in dietary intake of vitamin K will have less effect on the body's vitamin K storage and (2) to reduce the patient's sensitivity to the VKA especially when the patient in question has the “sensitive” VKORC1 genetic polymorphism. The compositions of the present invention are also combined into a single dosage form in order to eliminate the possibility that patients will take one component and not the other. Administration as a single dose avoids the dangers of administering two antagonistic medications as separate formulations.

### DETAILED DESCRIPTION OF PREFERRED EMBODIMENTS

The present invention relates to compositions administered in a single dose that comprise both vitamin K and one or more VKAs. The VKAs can be the R-isomer of warfarin, the R-isomer of phenprocoumon, racemic phenprocoumon, or a mixture of these.

One aspect of the present invention is a composition for use in oral anticoagulant therapy comprising low doses of vitamin K in combination with a vitamin K antagonist. The vitamin K antagonist may be the R isomer of warfarin, the R isomer of phenprocoumon, a racemic phenprocoumon mixture, or a mixture of these. The oral anticoagulant composition is administered as a single dose of the vitamin K in combination with the VKA, and the vitamin K is administered in a low dosage. Low dosage usually means from about 25 mcg per day to about 1000 mcg per day, preferably in the range of about 100 mcg per day to about 200 mgc per day.

In order to avoid or reduce the influence of CYP 2C9 Polymorphisms, the claimed compositions include the R-isomer of warfarin, the R-isomer of phenprocoumon, and/or the phenprocoumon racemic mixture. The R-isomers of warfarin and phenprocoumon have been studied in terms of their potency and pharmacokinetics, but these two products have not been used in clinical practice, nor has either isomer been approved for marketing as a therapeutic agent. Racemic phenprocoumon is available in some countries but not in the U.S.

Drug interactions are substantially less significant with phenprocoumon than with warfarin and the effect of CYP 2C9 polymorphisms is minimal. (Sconce, et al. 2005; Oldenburg, J., 2005; O'Reilly, et al., 1979). In addition, alterations in 2C9 activity may have even less effect on the elimination of the R-isomer of phenprocoumon than on the racemic mixture. (Schalekam, et al. 2004; Kirchheiner, et al. 2004; Ufer 2005). This is true in part because approximately 40% of phenprocoumon is eliminated as the parent drug and because other hepatic enzymes are involved in metabolizing phenprocoumon and its isomers. (Toon, et al. 1985). Consequently, even though the racemic phenprocoumon offers an advantage over warfarin, the phenprocoumon R-isomer offers an additional advantage over racemic phenprocoumon.

The administration of a daily low dose of vitamin K (approximately 100 mcg) will minimize the variability in sensitivity to VKAs that results from the 3 VKORC1 genetic polymorphisms. This principle is based on the fact that when administering two agents that are antagonistic to each other (such as vitamin K and a VKA), the administered vitamin K will partially counteract the increased sensitivity to the VKA that is seen in those patients with a VKA “sensitive” VKORC1 genotype. By contrast, in those patients with a VKA “resistant” VKORC1 genotype a much higher dose of the VKA is required and the low dose of vitamin K will have little effect on the response to the large VKA dose. Therefore, the low dose vitamin K will moderate the response of “sensitive” patients (who typically have unstable INR responses) but the vitamin K will not substantially interfere with the effective VKA dose in “resistant” patients. Therefore, those patients with a “sensitive” VKORC1 genotype will demonstrate less VKA sensitivity and improved (more stable) INR response.

As already discussed, use of the R-isomer of warfarin, the R-isomer of phenprocoumon and/or racemic phenprocoumon will substantially reduce significant drug interactions with the VKAs. Many—if not most—of the more severe drug interactions with warfarin are due to either enhancement or inhibition of the activity of the CYP 2C9. (Lewis, et al. 1974; O'Reilly, et al. 1980; O'Reilly, et al. 1979; Ansell, et al. 2004). Avoiding or minimizing the impact of drug interactions is achieved by using the identified VKA products that are unaffected or minimally affected by CYP 2C9 enzyme metabolism. Furthermore, even though there are drug interactions that alter the activity of the enzymes that metabolize the R isomer of warfarin, these interactions have only a modest effect on the INR. (Ansell, et al. 2004).

In addition to circumventing or minimizing the role of 2C9 metabolism in the metabolism of the VKA, the three selected VKAs all have longer half-lives than the S-isomer of warfarin. The advantage of having a long half-life is two fold: (1) an occasionally missed dose should have limited impact on the INR and (2) drug interactions that result from altered metabolism of the VKA should evolve significantly more slowly, thereby allowing the clinician more time to identify the interaction and make appropriate dosage adjustments. Racemic phenprocoumon has an elimination half-life of approximately 156 hrs, which is 4 to 5 times that of the half-life of racemic warfarin (approximately 36 hrs.). (O'Reilly 1976). The R-isomer of warfarin has only a fraction of the potency of the S-isomer which also is metabolized more quickly, with a half-life of about 30 hrs while the R-isomer has a half-life of about 40 hrs. (Lewis 1974; O'Reilly, et al. 1980). However, because of the inter-patient variability in the metabolism of the S-isomer, in some patients the half-life of the S-isomer may be less than one-half that of the R-isomer. Therefore, by multiple mechanisms, the R-

isomer of warfarin, racemic phenprocoumon, and the R-isomer of phenprocoumon should have fewer and less severe drug interactions than currently occur with warfarin.

It is also well established that changes in one's dietary intake of vitamin K can have a significant effect on the response to the VKA. Specifically, a significant increase in vitamin K intake can partially reverse the effect of the VKA, thereby inducing a fall in the INR and an increase in the risk of clotting (thrombosis). Alternatively, a reduction in the vitamin K intake will lead to an enhanced effect of the VKA, thereby inducing an increase in the INR and an increase in the risk of bleeding. For decades, clinicians have typically advised patients taking a VKA to avoid those foods that contain large amounts of vitamin K. Certainly, it seems reasonable to instruct patients on a VKA to avoid foods with substantial vitamin K because vitamin K can counteract the effect of the VKA medication. Restricting vitamin K intake, however, actually serves to make the patient more sensitive to the VKA. This is especially true in those patients who have a “sensitive” VKORC1 polymorphism. Although it may at first seem counter-intuitive, the administration of a daily dose of vitamin K reduces the VKA sensitivity of those with a “sensitive” VKORC1 polymorphism. In addition, the daily administration of low dose vitamin K will increase the total amount of vitamin K in the body. With a higher total amount of vitamin K in the body, any change in a day's intake of vitamin K becomes less critical because any daily change in vitamin K intake would represent a proportionally smaller change in the body's vitamin K stores.

Because each of the current oral coagulation compositions will contain a VKA and vitamin K itself, it is critically important that the two components be combined in a single dosage form. Administering a vitamin K supplement as a separate product (as described in Example 3 below) presents the risk that the patient may run out of either the vitamin K or the VKA prescription. This potential is increased by the fact that the available vitamin K products are available in 100 tablet quantities while most prescription programs will allow 30-day or 90-day prescriptions. If a patient runs out of the vitamin K (or forgets to take their dose), then the effect of the VKA will be enhanced and the INR will rise and create an increased risk of bleeding. Alternatively, if one misses doses of the VKA but continues to take the vitamin K dose, the vitamin K supplement will cause the INR to fall faster and lower, thereby increasing the risk of a blood clot. Lastly, the only pharmaceutical grade form of vitamin K for oral administration is available in the U.S. in only a 5 mg (5,000 mcg) tablet. This tablet contains approximately 50 times as much vitamin K as is required for a daily supplement and actually is enough to totally reverse the effects of the VKA in most patients. Therefore, clinicians wishing to use a daily dose of vitamin K to stabilize the INR must use a dietary supplement which is available in 100 mcg tablets. However, these products are considered “dietary supplements” and are not subject to—nor do they have to meet—the quality control standards and the content verification standards that are required of a pharmaceutical product. Therefore, in order to be confident that the patient is actually receiving the desired amount of vitamin K on a daily basis, and in order to avoid the dangers of administering two antagonistic medications as separate formulations, it is imperative that a pharmaceutical quality combination product be made available.

The current compositions are preferably formed as tablets containing an effective amount of the vitamin K and one of the aforementioned VKAs. These products, since they have the same mechanism of action as warfarin, are used for any and all indications in which oral anticoagulation is indicated. These indications include, but are not limited to, prevention of blood clots that can cause deep vein thrombosis, pulmonary embolism, stroke, heart attacks, prosthetic heart valve thrombosis, and other clot-related conditions.

The compositions are preferably administered orally at a range of one-half tablet per day to multiple tablets per day. Rarely it may be necessary to administer more than 2 tablets daily. Because of the long half-life of these agents and their mechanism of action, once daily dosing should be appropriate for each agent.

The administration of these compositions requires close monitoring of the INR. The INR should be monitored frequently, such as every 1 to 3 days, when the medication is started. The dose of the medication should be adjusted based on the rate of rise in the INR and the amount of medication given in the previous several days. The dose of the medication is continually adjusted based on the INR result until a stable daily dose is determined for a given patient. Once the correct dose for the patient has been thusly determined, then the INR monitoring interval is increased progressively up to a maximum of approximately four weeks. If the dose has to be adjusted at a later day, then the more frequent INR monitoring is resumed until the correct dose is again determined and the monitoring intervals again extended to four week intervals. In addition to the INR monitoring, most clinicians also monitor the patient's blood level of hemoglobin and hematocrit as an indirect method to evaluate the patient for any unrecognized bleeding that may be occurring. As mentioned previously, bleeding is the most significant complication of anticoagulation therapy but it usually is rather uncommon as long as the INR is maintained within the desired therapeutic range.

### Example 1

### Tablet Production

Vitamin K is commercially available from Aton Pharmaceuticals (Lawrenceville, N.J.) as well as other commercial sources. Aton Pharm. currently markets Mephyton® 5 mg tablets. Phenprocoumon also is commercially available from Roche Pharmaceuticals (Basel, Switzerland) and other sources. R-isomers of warfarin and phenprocoumon also are commercially available through Aldrich Chemical (St. Louis, Mo.) and other sources, but the GMP requirements may require the independent separation of the R-isomers from the racemic mixture. Although several methods have been described for separating the R-isomers of coumarins from the racemic mixture, the method with the greatest yield appears that described previously in U.S. Pat. Nos. 5,856,525 and 5,686,631. This process involves oxidation of the VKA to a dehydro form which then is subjected to asymmetric hydrogenation. The R-isomer can then be recrystallized from a water/acetone source with reportedly greater than 98% enatiomeric purity as determined by high pressure liquid chromatography (“HPLC”).

Appropriate amounts of vitamin K and appropriate amounts of the selected VKAs (described in Example 2 below), are combined in the same mixture for compression into tablet formulations. Appropriate excipients are utilized as necessary for binders, glidants (flow aids), and lubricants to ensure efficient tabletting. Disintegrants may be required to assure that the tablets break up in the digestive tract. A suitable coating is applied to improve product stability and avoid unwanted taste. Multifunctional direct compression excipients (UICEL-XL and UICEL-A/102) are available and may be used (De La Luz Reus Medina and Kumar Vijay 2006). Otherwise, example binders include, but are not limited to, lactose powder, dibasic calcium phosphate, sucrose, corn starch, and microcrystalline cellulose, including hydroxymethyl cellulose. Disintegrants, which need to hydrate readily in the gastrointestinal tract, include such agents as starch and cellulose, which also may be used as binders. A lubricant, which includes but is not limited to, stearic acid, hydrogenated oil, and sodium stearyl fumarate is used as needed to help the tablet be more easily ejected from the die. A polymer or polysaccharide base is used for tablet coating to improve shelf life and prevent unpleasant taste. Pigments or dyes should not be used because with VKA therapy, allergy to the dye is often confused as allergy to the VKA.

Tablets are produced in an appropriately approved manufacturing facility as disc shaped by direct compression at a pressure between 0.82 and 1.63 MPa (Megapascal units, which equals 145 psi). Tablets can be scored on one side in order to allow for easy breakage and dosing with half tablets.

### Example 2

### Active Ingredients

The recommended dietary allowance for vitamin K is approximately 70 mcg. In an example study, 100 mcg of vitamin K with warfarin was used. The dose was increased to 200 mcg in two patients who failed to respond adequately to the 100 mcg dose. In addition, the administration of a VKA often requires that the patient take a half tablet on some days while other patients may require 1.5 or 2 tablets on other days. Consequently, the workable range of vitamin K per tablet is 10 mcg to 600 mcg, the preferred range is 25 mcg to 400 mcg, and the most preferred is 50 mcg to 200 mcg.

The R-isomer of warfarin is reported to have only about one-fifth the potency of the S-isomer, but the R isomer has a longer half-life than the S-isomer. Consequently, the plasma concentration of the R-isomer may accumulate to a greater degree than does the S-isomer such that with racemic warfarin therapy the R-isomer may contribute slightly more VKA activity than what one might expect based on the one-fifth potency. Because the average maintenance dose of racemic warfarin is approximately 5 mg per day, the average daily dose of the R-isomer is approximately 25 mg per day. Therefore, the workable range of the R-isomer of warfarin per tablet is 1 mg to 100 mg, the preferred range is 5 mg to 75 mg, and the most preferred is 10 mg to 50 mg.

The mean daily dose of phenprocoumon in one sample of patients was approximately 2.2 mg with a range from 0.6 mg to 4.2 mg. The currently available tablet of the racemic phenprocoumon contains 3 mg, which is an excessive dose for most patients and requires a dosing regimen of half-doses and omitted doses on certain days. Because low dose vitamin K can be expected to increase the phenprocoumon dose only slightly in some patients, the mean daily dose of phenprocoumon will remain below 3 mg. Therefore, the workable range of the racemic phenprocoumon is 0.4 mg to 6 mg, the preferred range is 0.6 mg to 4.5 mg, and the most preferred is 1.0 mg to 2.0 mg.

The relative potency requires further clinical study but in the absence of convincing data that a difference exists between the potency of the R and S isomer, a slightly wider dosing range is used for the R-isomer. Therefore, the workable range of the R-isomer of phenprocoumon is 0.1 mg to 20 mg, the preferred range is 0.2 mg to 10 mg, and the most preferred is 0.4 mg to 6.0 mg.

### Example 3

### Combined Low Dosage of Vitamin K

It was tested whether daily vitamin K supplementation would improve stability of the INR in patients who had very unstable INRs. The presence of unstable INRs would suggest that these patients had low vitamin K body stores and/or had a “sensitive” VKORC1 genotype. The VKORC1 status itself was not assessed because the test was done prior to the VKORC1 gene being identified. INR values were collected for several months before and after daily vitamin K was started in eight patients with very unstable INR values. It was found that the INRs of these patients fluctuated less after they started a daily vitamin K supplement and the percent of INRs that were in range more than doubled after the patients started taking vitamin K. (Reese, et al. 2005) The use of a daily vitamin K supplement to moderate VKA sensitivity in “sensitive” VKORC1 genotype patients had not been studied or even suggested in the clinical literature. Also, the idea of combining a daily low dose vitamin K supplement to improve INR stability with individual VKA isomers also had not be studied or suggested. However, the results indicate that combining a daily low dose vitamin K supplement did improve INR stability.

### REFERENCES CITED

The entire content of each of the following documents is hereby incorporated by reference.

### U.S. PATENT DOCUMENTS

U.S. Patents

| | |
|---|---|
| U.S. Pat. No. 2,427,578 | Stahmann et al. |
| U.S. Pat. No. 5,041,430 | Addicks et al. |
| U.S. Pat. No. 5,686,631 | Li et al. |
| U.S. Pat. No. 5,856,525 | Li et al. |

### OTHER PATENT DOCUMENTS

Canadian Patent No. 2388103 Bertling

### OTHER PUBLICATIONS

Ansell, J., et al. Chest 2004; 126:204s-233s.
De La Luz Reus Medina and Kumar Vijay. International Journal of Pharmaceutics 2006; 322:31-35.
Kirchheiner, J., et al. Pharmacogenetics 2004; 14:19-26.
Lewis, R. J., et al. J Clin Invest. 1974; 1607-17.
Oldenburg, J., Vitamin K intake and stability of oral anticoagulant treatment. Thromb Haemost 2005; 93:799-800.
O'Reilly, R. A. Ann Rev Med. 1976; 27:245-61.
O'Reilly, R. A., et al. Racemic warfarin and trimethoprim-sulfamethoxazole interaction in humans. Ann Intern Med 1979; 91:34-36.
O'Reilly, R. A., et al. J Clin Invest. 1980; 6:746-53.
Reese, A. M., et al. Pharmacotherapy 2005; 25(12):1746-51.
Schalekamp, T., et al. Clin Pharmacol Ther 2004; 76:409-417.
Sconce, E., et al. Patients with unstable control have a poorer dietary intake of vitamin K compared to patients with stable control of anticoagulation. Thromb Haemost 2005; 93: 872-5.
Toon, S., et al. J Pharm Sci 1985; 74:1037-40.
Ufer, M. Clin Pharmacol Ther 2005; April, 335.
White, H. D., et al. Arch Intern Med. 2007; 167:239-245.

What is claimed is:

1. A composition for use in anticoagulant therapy comprising:
Vitamin K; and
a Vitamin K antagonist,
wherein the vitamin K is present in a low dosage and wherein the Vitamin K and the Vitamin K antagonist are combined such that the composition is administered in a single dosage.

2. The composition of claim 1 wherein the Vitamin K antagonist is the R-isomer of warfarin, the R-isomer of phenprocoumon, racemic phenprocoumon, or a mixture thereof.

3. The composition of claim 1 wherein the vitamin K is present in an amount of about 50 mcg to about 200 mcg.

4. The composition of claim 1 wherein the vitamin K antagonist is an R-isomer of warfarin and wherein the R-isomer of warfarin is present in an amount of about 10 mg to about 50 mg.

5. The composition of claim 1 wherein the vitamin K antagonist is an R-isomer of phenprocoumon and wherein the R-isomer of phenprocoumon is present in an amount of about 0.4 mg to about 6 mg.

6. The composition of claim 1 wherein the vitamin K antagonist is a racemic phenprocoumon and wherein the racemic phenprocoumon is present in an amount of about 1 to about 2 mg.

7. The composition of claim 1 further comprising one or more binders, disintegrants, lubricants, or coatings.

8. The composition of claim 1 wherein the single dosage is suitable for oral administration.

9. The composition of claim 8 wherein the single dosage is a tablet for oral administration.

10. A method for treating a patient in need of anticoagulant therapy comprising:
administering a composition comprising Vitamin K and a Vitamin K antagonist,
wherein the vitamin K is present in a low dosage and wherein the Vitamin K and the Vitamin K antagonist are combined such that the composition is administered in a single dosage.

11. The method of claim 10 wherein the Vitamin K antagonist is the R-isomer of warfarin, the R-isomer of phenprocoumon, racemic phenprocoumon, or a mixture thereof.

12. The method of claim 1 wherein the vitamin K is present in an amount of about 50 mcg to about 200 mcg.

13. The method of claim 1 wherein the vitamin K antagonist is an R-isomer of warfarin and wherein the R-isomer of warfarin is present in an amount of about 10 mg to about 50 mg.

14. The method of claim 1 wherein the vitamin K antagonist is an R-isomer of phenprocoumon and wherein the R-isomer of phenprocoumon is present in an amount of about 0.4 mg to about 6 mg.

15. The method of claim 1 wherein the vitamin K antagonist is a racemic phenprocoumon and wherein the racemic phenprocoumon is present in an amount of about 1 to about 2 mg.

16. The method of claim 1 wherein the composition further comprises one or more binders, disintegrants, lubricants, or coatings.

17. The method of claim 1 wherein the administering of the single dosage is by oral administration.

18. The method of claim 17 wherein the single dosage is a tablet for oral administration.

19. The method of claim 18 wherein the composition is administered in an amount of from one half to two tablets per day.

20. The method of claim 18 wherein the composition is administered in an amount that is adjusted based on the rate of rise of INR in the patient.

21. A pharmaceutical formulation for use in anticoagulant therapy comprising:
Vitamin K;
a Vitamin K antagonist selected from the group consisting of an R-isomer of warfarin, an R-isomer of phenprocoumon, a racemic phenprocoumon, and a mixture thereof; and
one or more binders, disintegrants, lubricants, or coatings,
wherein the vitamin K in an amount of about 50 mcg to about 200 mcg and wherein the Vitamin K and the Vitamin K antagonist are combined such that the composition is administered in a single dosage.

22. The pharmaceutical formulation of claim 21, wherein the vitamin K antagonist is an R-isomer of warfarin and wherein the R-isomer of warfarin is present in an amount of about 10 mg to about 50 mg.

23. The pharmaceutical formulation of claim 21 wherein the vitamin K antagonist is an R-isomer of phenprocoumon and wherein the R-isomer of phenprocoumon is present in an amount of about 0.4 mg to about 6 mg.

24. The pharmaceutical formulation of claim 21 wherein the vitamin K antagonist is a racemic phenprocoumon and wherein the racemic phenprocoumon is present in an amount of about 1 to about 2 mg.

25. The pharmaceutical formulation of claim 21 wherein the single dosage is a tablet for oral administration.
